(19) United States
(12) Patent Application Publication
(10) Pub. No.: US 2023/0139637 A1
(43) Pub. Date: May 4, 2023

(54) VITAL SIGN MONITORING VIA REMOTE SENSING ON STATIONARY EXERCISE EQUIPMENT

(71) Applicant: Arizona Board of Regents on behalf of Arizona State University, Scottsdale, AZ (US)

(72) Inventors: Daniel W. Bliss, Phoenix, AZ (US); Yu Rong, Phoenix, AZ (US); Arindam Dutta, Tempe, AZ (US); Alex Chiriyath, Tempe, AZ (US)

(21) Appl. No.: 17/801,543

(22) PCT Filed: Mar. 31, 2021
(86) PCT No.: PCT/US2021/025106

§ 371 Date: Aug. 23, 2022

Related U.S. Application Data
(60) Provisional application No. 63/002,730, filed on Mar. 31, 2020.

(51) Int. Cl. A61B 5/05; A61B 5/11; A61B 5/00

(52) U.S. Cl. CPC A61B 5/05; A61B 5/1114; A61B 5/1118; A61B 5/6895; A61B 5/7203; A61B 5/7267; A61B 5/7275

(57) ABSTRACT

Vital sign monitoring via remote sensing on stationary exercise equipment is provided. A new non-contact approach described herein uses radio frequency (RF) radar (e.g., ultra-wide band (UWB) radar) to remotely monitor vital sign information (such as heartbeat and breathing) and human activity information of subjects using stationary exercise equipment. In some embodiments, a radar sensor captures micro-scale chest motions (corresponding to the vital sign information) as well as macro-scale body motions (corresponding to movements from exercise). A signal processor receives radar signals from the radar sensor and processes the radar signals to reconstruct vital sign information from the micro-scale chest motions and/or human activity information from the macro-scale body motions using a joint vital sign-motion model, which can be trained using machine learning and other approaches.

### RELATED APPLICATIONS

This application claims the benefit of Provisional Patent application serial number 63/002,730, filed Mar. 31, 2020, the disclosure of which is hereby incorporated herein by reference in its entirety.

### FIELD OF THE DISCLOSURE

This disclosure relates to remote vital sign detection during exercise.

### BACKGROUND

Remote sensing of physiological parameters, such as heartbeat and breathing, has a number of uses. There is a strong demand for providing accurate and timely vital sign information in a convenient and less expensive way during everyday activities, including exercise. Existing approaches can cause discomfort and may not always be suitable for monitoring of physiological parameters during exercise.

Common methods of exercise monitoring, such as electrocardiograms (ECGs) and photoplethysmography (PPG) sensors, require direct contact with the human body to measure vital signs. For example, chest strap heart rate sensors and other wearable devices can provide accurate vital sign measurements but may cause discomfort during exercise. Additionally, some stationary exercise equipment has one or more contact sensors that require continuous contact with the user (such as through palms) during exercise, which is also uncomfortable and can even cause injury due to limiting user movement.

Non-contact remote sensing-based exercise monitoring methods do not suffer from these issues. For these reasons, non-contact approaches for exercise monitoring have tremendous utility when compared to current commercial technology. Previous approaches have focused on using a camera or other optical sensor to find heart rate through changes in skin tone indicating changes in blood volume due to cardiac activity. However, these approaches encounter issues under inconsistent lighting conditions, require naked skin to be visible to the optical sensor, are not robust against different skin tones, and face issues of privacy and security.

### SUMMARY

Vital sign monitoring via remote sensing on stationary exercise equipment is provided. A new non-contact approach described herein uses radio frequency (RF) radar (e.g., ultra-wide band (UWB) radar) to remotely monitor vital sign information (such as heartbeat and breathing) and human activity information of subjects using stationary exercise equipment. In some embodiments, a radar sensor captures micro-scale chest motions (corresponding to the vital sign information) as well as macro-scale body motions (corresponding to movements from exercise). A signal processor receives radar signals from the radar sensor and processes the radar signals to reconstruct vital sign information from the micro-scale chest motions and/or human activity information from the macro-scale body motions using a joint vital sign-motion model, which can be trained using machine learning and other approaches.

An exemplary embodiment provides a method for monitoring vital signs of a subject using exercise equipment. The method includes receiving a radar return signal measuring a region of interest of the subject; processing the radar return signal to produce micro-Doppler data of the region of interest; and applying a joint motion-vital sign model to the micro-Doppler data to estimate vital sign information of the subject.

Another exemplary embodiment provides a vital sign monitoring system. The vital sign monitoring system includes a RF radar sensor and a signal processor. The signal processor is configured to receive a radar return signal from the RF radar sensor; perform a micro-Doppler analysis of a region of interest using a joint motion-vital sign model; and extract vital sign information of one or more subjects based on the micro-Doppler analysis.

Those skilled in the art will appreciate the scope of the present disclosure and realize additional aspects thereof after reading the following detailed description of the preferred embodiments in association with the accompanying drawing figures.

### BRIEF DESCRIPTION OF THE DRAWING FIGURES

The accompanying drawing figures incorporated in and forming a part of this specification illustrate several aspects of the disclosure, and together with the description serve to explain the principles of the disclosure.

FIG. 1A is a schematic diagram of an exemplary exercise equipment which includes a vital sign monitoring system.

FIG. 1B is a schematic block diagram of the vital sign monitoring system of FIG. 1A.

FIG. 2 is a schematic block diagram of an exemplary process for monitoring vital signs of a subject using exercise equipment.

FIG. 3 is a schematic block diagram of an exemplary process for monitoring vital sign information from a subject using the exercise equipment of FIGS. 1A and 1B.

FIG. 4A is a graphical representation of a reference heart rate signal.

FIG. 4B is a graphical representation of a radar return signal received by an exemplary embodiment which corresponds to the reference heart rate signal of FIG. 4A.

FIG. 5A is a graphical representation of example training data collected from a subject performing an unsupervised (e.g., free-living) workout.

FIG. 5B is a graphical representation of example testing data collected from the subject performing an unsupervised workout.

FIG. 6 is a block diagram of the vital sign monitoring system according to embodiments disclosed herein.

### DETAILED DESCRIPTION

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that when an element such as a layer, region, or substrate is referred to as being “on” or extending “onto” another element, it can be directly on or extend directly onto the other element or intervening elements may also be present. In contrast, when an element is referred to as being “directly on” or extending “directly onto” another element, there are no intervening elements present. Likewise, it will be understood that when an element such as a layer, region, or substrate is referred to as being “over” or extending “over” another element, it can be directly over or extend directly over the other element or intervening elements may also be present. In contrast, when an element is referred to as being “directly over” or extending “directly over” another element, there are no intervening elements present. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Relative terms such as “below” or “above” or “upper” or “lower” or “horizontal” or “vertical” may be used herein to describe a relationship of one element, layer, or region to another element, layer, or region as illustrated in the Figures. It will be understood that these terms and those discussed above are intended to encompass different orientations of the device in addition to the orientation depicted in the Figures.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

Vital sign monitoring via remote sensing on stationary exercise equipment is provided. A new non-contact approach described herein uses radio frequency (RF) radar (e.g., ultra-wide band (UWB) radar) to remotely monitor vital sign information (such as heartbeat and breathing) and human activity information of subjects using stationary exercise equipment. In some embodiments, a radar sensor captures micro-scale chest motions (corresponding to the vital sign information) as well as macro-scale body motions (corresponding to movements from exercise). A signal processor receives radar signals from the radar sensor and processes the radar signals to reconstruct vital sign information from the micro-scale chest motions and/or human activity information from the macro-scale body motions using a joint vital sign-motion model, which can be trained using machine learning and other approaches.

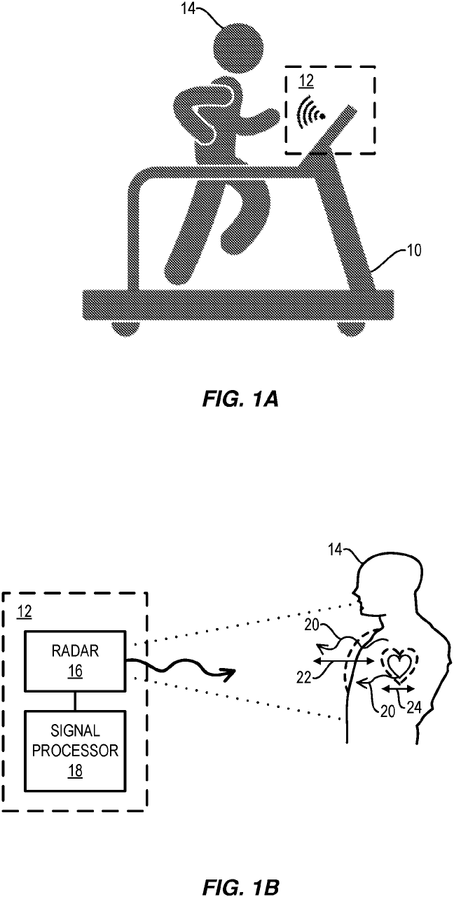

FIG. 1A is a schematic diagram of an exemplary exercise equipment 10 which includes a vital sign monitoring system 12. The vital sign monitoring system 12 provides a non-contact approach to tracking the activity of the human body of a subject 14 during exercise and providing information on vital signs and other physiological factors.

In an exemplary aspect, the exercise equipment 10 is stationary exercise equipment, such as a treadmill, stationary bicycle, elliptical trainer, stepper machine, rowing machine, weight machine, etc. The vital sign monitoring system 12 may be placed on one or more machines in an exercise area. For example, each of multiple exercise equipment 10 in a given exercise area may incorporate the vital sign monitoring system 12. In other examples, a single vital sign monitoring system 12 may monitor vital signs and other activities of multiple subjects 14 on different exercise equipment 10. For example, the vital sign monitoring system 12 can be placed in an exercise area (e.g., on a wall, a ceiling, exercise equipment 10, etc.) and used to provide vital sign and/or activity information of one or more subjects 14.

FIG. 1B is a schematic block diagram of the vital sign monitoring system 12 of FIG. 1A. The vital sign monitoring system 12 includes a radar sensor 16 (e.g., a UWB radar sensor) to remotely measure the motion of the body (e.g., macro and micro) of the subject 14. The vital sign monitoring system 12 also includes a signal processor 18 which processes radar signals received by the radar sensor 16 to determine one or more vital signs of the subject 14.

In an exemplary aspect, the radar signals can be processed by the signal processor 18 to measure the rate of chest displacement due to breathing and heartbeat. From these measurements, the heart rate and/or respiration rate of the subject 14 can be obtained. In this regard, embodiments may extract vital sign information of the subject 14, which can include, but is not limited to, a heart rate, a respiration rate, a heart signal, a respiration signal, a heart rate variability, and inter-beat information (e.g., statistics of inter-beat intervals, which can be used to predict cardiac distress).

In some embodiments, the signal processor 18 can also determine other activity-based information. For example, embodiments can extract activity data such as gait, step rate, type of activity engaged in (e.g., jogging, rowing, weightlifting, etc.), asymmetries in body motion, and so on. Depending on the application, embodiments can identify gross activities, such as jogging, rowing, weightlifting, etc. (e.g., when the radar sensor measures an exercise area) as well as more subtle distinctions between activity subtypes, such as a walk, jog, or run (e.g., when the radar is attached to a treadmill). The activity data may further be analyzed to determine signs of distress or fatigue in the subject 14 (e.g., a change in motion rates or asymmetrical motion can indicate risks of injury), to guide recovery of an injured subject 14, and so on.

In an exemplary aspect, the radar sensor 16 is coupled to the signal processor 18, which is used to estimate vital sign (e.g., heart rate) and/or macro body motion information of the subject 14 during exercise (or other activities) using a joint motion-vital sign model. A radar return signal received by the radar sensor 16 includes an RF response of human motion. The RF response of the subject 14 (including vital sign motion) is modeled as a superposition of responses from discrete, dynamic scattering centers 20, which may be from various body parts (e.g., chest movement from respiratory activity 22 and cardiac activity 24, as well as macro motion of the subject 14 as it engages in exercise or other activities).

The radar sensor 16 includes a radar receiver to receive the radar return signal and may further include a radar transmitter which emits a radar signal. The radar sensor 16 can receive (and in some embodiments emit) a radar return signal in any RF band, such as terrestrial radio frequencies, gigahertz (GHz) bands, terahertz bands, microwave bands, etc. In some examples, the radar sensor 16 operates on an impulse signaling scheme with a wide bandwidth and a center frequency greater than 5 GHz (e.g., a center frequency of 7.3 GHz with a bandwidth of 1.4 GHz). The radar sensor 16 may have a detection range of 6 meters (m) or greater, depending on conditions and RF parameters.

In an exemplary aspect, an i-th scattering center 20 is parameterized by reflectivity coefficient pᵢ(t) and radial distance dᵢ(t) from the radar sensor 16, which vary as a function of time t. The received composite signal is modeled as follows:

$$y \left(τ , t\right) = {\sum }_{i}^{N} {ρ}_{i} \left(t\right) p \text{} \left(τ - {τ}_{{d}_{i}} \left(t\right)\right)$$

$$= {\sum }_{i}^{N} {ρ}_{i} \left(t\right) p \left(τ - 2 \frac{{d}_{i} \left(t\right)}{c}\right)$$

where N is the number of scattering centers and p(τ) is the transmitted pulse. c denotes the speed of light. Note that t and τ are two different time scales. The former is often referred to as a slow-time sampling interval and is related to the pulse repetition interval. The latter time scale is referred as a fast-time sampling interval and is often associated with an analog-to-digital converter (ADC) sampling interval providing distance information.

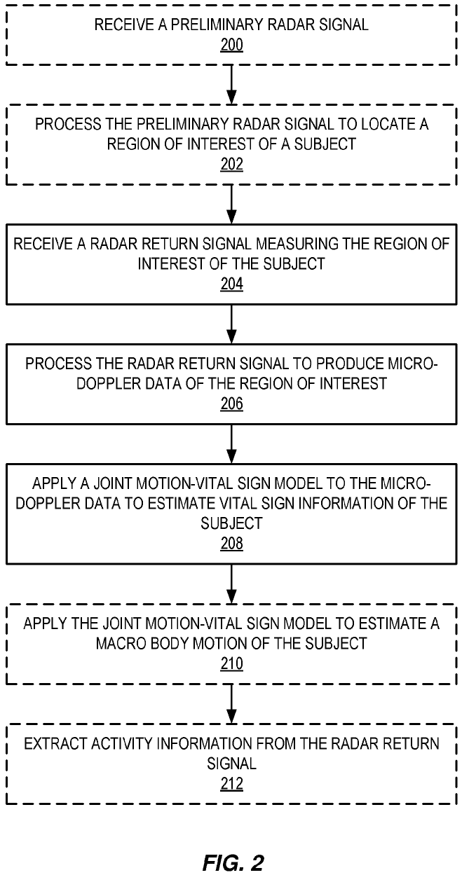

FIG. 2 is a schematic block diagram of an exemplary process for monitoring vital signs of a subject using exercise equipment (e.g., the exercise equipment 10 of FIG. 1A). The process optionally begins with receiving a preliminary radar signal (block 200). The process optionally continues with processing the preliminary radar signal to locate a region of interest of a subject (e.g., a human subject) (block 202), which can also be considered a calibration of the radar sensor 16 of FIGS. 1A and 1B. The process continues with receiving a radar return signal measuring the region of interest of the subject (block 204). In an exemplary aspect, the radar return signal corresponds to a response from a single radar emitter.

The process continues with processing the radar return signal to produce micro-Doppler data of the region of interest (block 206). The process continues with applying a joint motion-vital sign model to the micro-Doppler data to estimate vital sign information of the subject (block 208). The process optionally continues with applying the joint motion-vital sign model to estimate a macro body motion of the subject (block 210). The process optionally continues with extracting activity information from the radar return signal (block 212).

As used herein, micro-Doppler data refers to a time series of radar return data that contains human motions quantitatively. The micro-Doppler data can include one or more micro-Doppler images, which are a way to visualize the motion information in the time series data. For example, in embodiments described herein, the subject is running on a treadmill. There is a baseline body motion in addition to varying body motions associated with exercise. Some of the motions of interest are vital motions (e.g., skin surface motions) from breathing and heartbeat. At one observation time, in the spectral domain, these different motions correspond to different frequency shifts and different intensities. At a next observation time, another of these motions is generated. The frequency shifts across different observation times change slightly and provide information of how the micro-motions change over time. These multiple observations are referred to herein as micro-Doppler data (which can include micro-Doppler images or measurements).

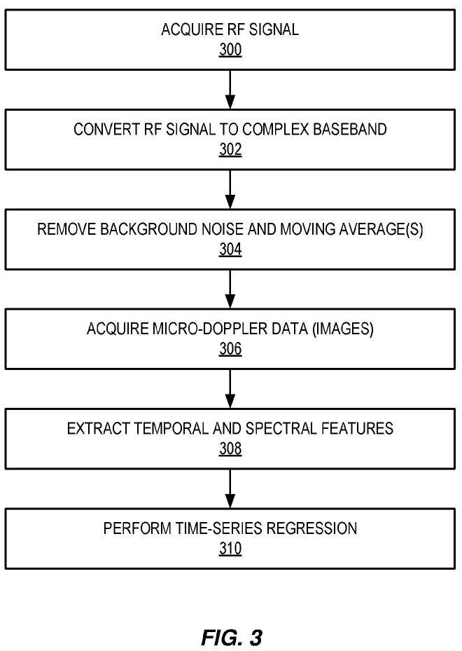

FIG. 3 is a schematic block diagram of an exemplary process for monitoring vital sign information from a subject using the exercise equipment of FIGS. 1A and 1B. The process begins with acquiring a RF signal (e.g., radar return signal) (block 300). The RF signal may be an UWB radar signal and may be emitted from one or multiple emitting antennas. Generally, the RF signal is emitted from a single radar transmitter (which may be part of or separate from the radar sensor 16 of FIG. 1B) and received over multiple antennas in order to better capture vital sign motions. The process continues with converting the RF signal to complex baseband (block 302). The process continues with mitigating clutter in the RF signal, such as by removing background noise using one or more moving averages (e.g., using a high-pass filtering approach which subtracts the moving average(s) from the RF signal) (block 304). The clutter mitigation removes or reduces artifacts in the RF signal that are not due to a gross body motion or vital sign motion of the subject (i.e., anything in the radar data not coming from the subject).

The process continues with processing the radar signal to acquire micro-Doppler data (e.g., one or more micro-Doppler images) of the region(s) of interest (block 306). The process continues with extracting temporal and spectral features from the micro-Doppler images in accordance with the joint motion-vital sign model (block 308). In this regard, the joint motion-vital sign model identifies a number of temporal and spectral features that are used to estimate vital sign and macro motion information of the subject. Such features include, but are not limited to, short-time energy, energy entropy, spectral centroid, spectral spread, spectral entropy, and spectral flux.

The process continues with using a time-series regression of the temporal and spectral features to reconstruct (e.g., measure or estimate) vital sign information of the subject (block 310). The vital sign information can include a heart rate, a respiration rate, a heart signal, a respiration signal, a heart rate variability, inter-beat information, etc.

In an exemplary aspect, an M5 rules regression model is used. The time-series regression can use data from an appropriate time frame, such as the previous 2 seconds. The M5 rules regression is a tree-based regression that fits a different linear model for every split. It should be noted that other regression models can also be used, such as a linear regression model.

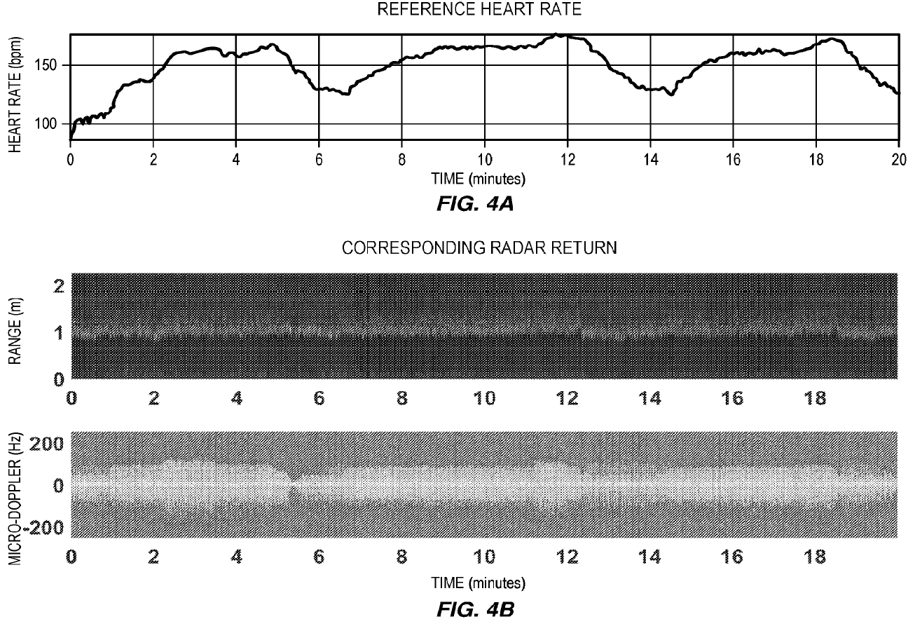

FIG. 4A is a graphical representation of a reference heart rate signal. The reference heart rate signal was measured by a contact heart rate sensor to demonstrate effectiveness of embodiments described herein.

FIG. 4B is a graphical representation of a radar return signal received by an exemplary embodiment which corresponds to the reference heart rate signal of FIG. 4A. FIG. 4B provides an illustration of a range image and a micro-Doppler image based on a radar return signal received and processed by the radar sensor 16 of FIG. 1B. While the range data alone may be insufficient to extract vital sign information, the heart rate information in the reference signal of FIG. 4A is reflected in the micro-Doppler data illustrated in the micro-Doppler image.

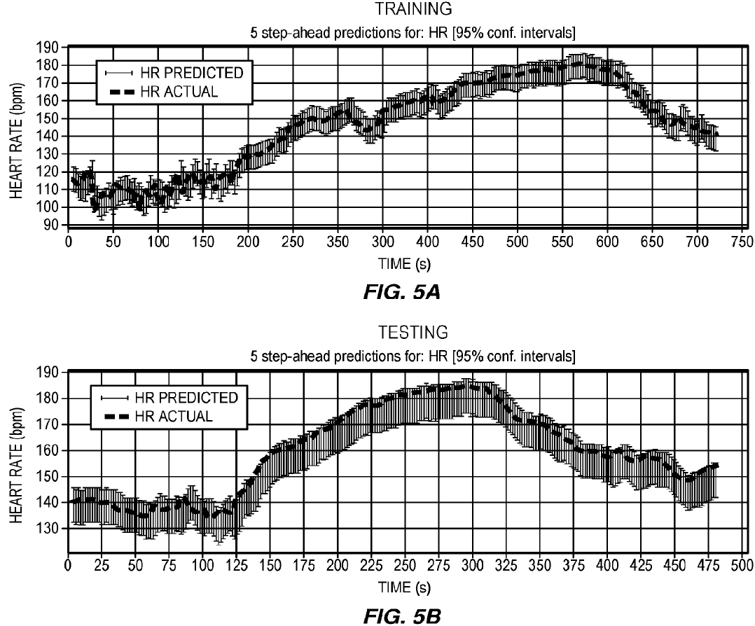

FIG. 5A is a graphical representation of example training data collected from a subject performing an unsupervised (e.g., free-living) workout. FIG. 5B is a graphical representation of example testing data collected from the subject performing an unsupervised workout. The estimated heart rate of the subject according to embodiments of the vital sign monitoring system 12 of FIGS. 1A and 1B is illustrated in black. The actual heart rate of the subject measured by a contact electrocardiogram (ECG) sensor is illustrated in gray. FIGS. 5A-5B illustrate the effectiveness of the approach described herein in measuring vital sign information, such as the heart signal.

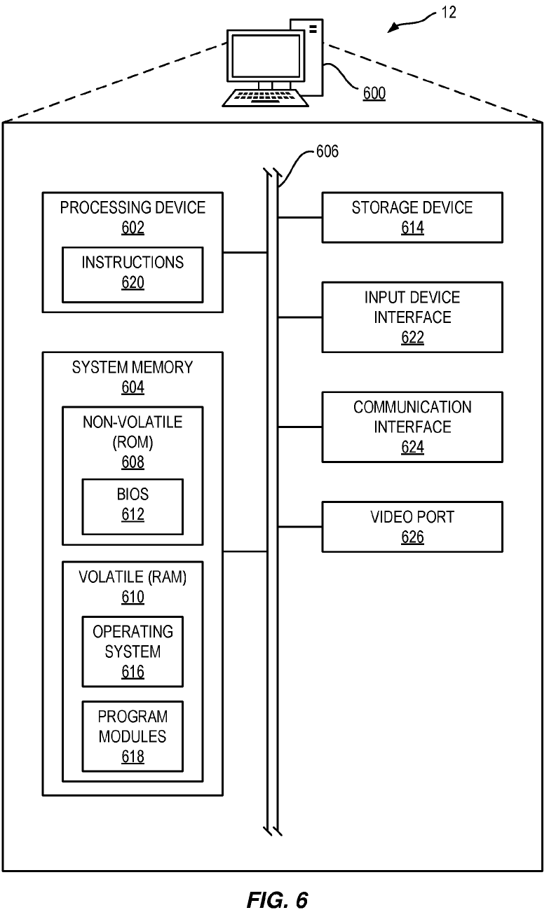

FIG. 6 is a block diagram of the vital sign monitoring system 12 according to embodiments disclosed herein. The vital sign monitoring system 12 includes or is implemented as a computer system 600, which comprises any computing or electronic device capable of including firmware, hardware, and/or executing software instructions that could be used to perform any of the methods or functions described above. In this regard, the computer system 600 may be a circuit or circuits included in an electronic board card, such as a printed circuit board (PCB), a server, a personal computer, a desktop computer, a laptop computer, an array of computers, a personal digital assistant (PDA), a computing pad, a mobile device, or any other device, and may represent, for example, a server or a user’s computer.

The exemplary computer system 600 in this embodiment includes a processing device 602 or processor, a system memory 604, and a system bus 606. The system memory 604 may include non-volatile memory 608 and volatile memory 610. The non-volatile memory 608 may include read-only memory (ROM), erasable programmable read-only memory (EPROM), electrically erasable programmable read-only memory (EEPROM), and the like. The volatile memory 610 generally includes random-access memory (RAM) (e.g., dynamic random-access memory (DRAM), such as synchronous DRAM (SDRAM)). A basic input/output system (BIOS) 612 may be stored in the non-volatile memory 608 and can include the basic routines that help to transfer information between elements within the computer system 600.

The system bus 606 provides an interface for system components including, but not limited to, the system memory 604 and the processing device 602. The system bus 606 may be any of several types of bus structures that may further interconnect to a memory bus (with or without a memory controller), a peripheral bus, and/or a local bus using any of a variety of commercially available bus architectures.

The processing device 602 represents one or more commercially available or proprietary general-purpose processing devices, such as a microprocessor, central processing unit (CPU), or the like. More particularly, the processing device 602 may be a complex instruction set computing (CISC) microprocessor, a reduced instruction set computing (RISC) microprocessor, a very long instruction word (VLIW) microprocessor, a processor implementing other instruction sets, or other processors implementing a combination of instruction sets. The processing device 602 is configured to execute processing logic instructions for performing the operations and steps discussed herein.

In this regard, the various illustrative logical blocks, modules, and circuits described in connection with the embodiments disclosed herein may be implemented or performed with the processing device 602, which may be a microprocessor, field programmable gate array (FPGA), a digital signal processor (DSP), an application-specific integrated circuit (ASIC), or other programmable logic device, a discrete gate or transistor logic, discrete hardware components, or any combination thereof designed to perform the functions described herein. Furthermore, the processing device 602 may be a microprocessor, or may be any conventional processor, controller, microcontroller, or state machine. The processing device 602 may also be implemented as a combination of computing devices (e.g., a combination of a DSP and a microprocessor, a plurality of microprocessors, one or more microprocessors in conjunction with a DSP core, or any other such configuration).

The computer system 600 may further include or be coupled to a non-transitory computer-readable storage medium, such as a storage device 614, which may represent an internal or external hard disk drive (HDD), flash memory, or the like. The storage device 614 and other drives associated with computer-readable media and computer-usable media may provide non-volatile storage of data, data structures, computer-executable instructions, and the like. Although the description of computer-readable media above refers to an HDD, it should be appreciated that other types of media that are readable by a computer, such as optical disks, magnetic cassettes, flash memory cards, cartridges, and the like, may also be used in the operating environment, and, further, that any such media may contain computer-executable instructions for performing novel methods of the disclosed embodiments.

An operating system 616 and any number of program modules 618 or other applications can be stored in the volatile memory 610, wherein the program modules 618 represent a wide array of computer-executable instructions corresponding to programs, applications, functions, and the like that may implement the functionality described herein in whole or in part, such as through instructions 620 on the processing device 602. The program modules 618 may also reside on the storage mechanism provided by the storage device 614. As such, all or a portion of the functionality described herein may be implemented as a computer program product stored on a transitory or non-transitory computer-usable or computer-readable storage medium, such as the storage device 614, non-volatile memory 608, volatile memory 610, instructions 620, and the like. The computer program product includes complex programming instructions, such as complex computer-readable program code, to cause the processing device 602 to carry out the steps necessary to implement the functions described herein.

An operator, such as the user, may also be able to enter one or more configuration commands to the computer system 600 through a keyboard, a pointing device such as a mouse, or a touch-sensitive surface, such as the display device, via an input device interface 622 or remotely through a web interface, terminal program, or the like via a communication interface 624. The communication interface 624 may be wired or wireless and facilitate communications with any number of devices via a communications network in a direct or indirect fashion. An output device, such as a display device, can be coupled to the system bus 606 and driven by a video port 626. Additional inputs and outputs to the computer system 600 may be provided through the system bus 606 as appropriate to implement embodiments described herein.

The operational steps described in any of the exemplary embodiments herein are described to provide examples and discussion. The operations described may be performed in numerous different sequences other than the illustrated sequences. Furthermore, operations described in a single operational step may actually be performed in a number of different steps. Additionally, one or more operational steps discussed in the exemplary embodiments may be combined.

Those skilled in the art will recognize improvements and modifications to the preferred embodiments of the present disclosure. All such improvements and modifications are considered within the scope of the concepts disclosed herein and the claims that follow.

What is claimed is:

1. A method for monitoring vital signs of a subject using exercise equipment, the method comprising:
receiving a radar return signal measuring a region of interest of the subject;
processing the radar return signal to produce micro-Doppler data of the region of interest; and
applying a joint motion-vital sign model to the micro-Doppler data to estimate vital sign information of the subject.

2. The method of claim 1, wherein the radar return signal is received in response to a single radar emitter.

3. The method of claim 1, further comprising applying the joint motion-vital sign model to estimate a macro body motion of the subject.

4. The method of claim 3, further comprising extracting activity information from the radar return signal.

5. The method of claim 4, wherein the activity information comprises at least one of a gait of the subject or a type of activity engaged in by the subject.

6. The method of claim 1, further comprising converting the radar return signal from radio frequency (RF) to a complex baseband.

7. The method of claim 6, further comprising removing background noise from the radar return signal after converting to the complex baseband.

8. The method of claim 1, wherein applying the joint motion-vital sign model to the micro-Doppler data comprises:
extracting a set of temporal and spectral features from the micro-Doppler data; and
performing a time-series regression of the set of temporal and spectral features.

9. The method of claim 1, further comprising producing the joint motion-vital sign model by:
training a prediction algorithm to estimate vital sign information at a plurality of activity rates; and
correcting the prediction algorithm using a contact sensor.

10. The method of claim 1, wherein the vital sign information comprises at least one of a heart rate, a respiration rate, a heartbeat waveform, or a respiration waveform.

11. The method of claim 1, further comprising:
receiving a preliminary radar signal before the radar return signal; and
processing the preliminary radar signal to locate the region of interest.

12. The method of claim 11, wherein processing the preliminary radar signal to locate the region of interest comprises identifying sets of micro-Doppler data indicating movement corresponding to chest movement of the subject.

13. A vital sign monitoring system, comprising:
a radio frequency (RF) radar sensor; and
a signal processor configured to:
receive a radar return signal from the RF radar sensor;
perform a micro-Doppler analysis of a region of interest using a joint motion-vital sign model; and
extract vital sign information of one or more subjects based on the micro-Doppler analysis.

14. The vital sign monitoring system of claim 13, wherein the vital sign information comprises at least one of a heart rate, a breathing rate, a heart signal, a breathing signal, a heart-rate variability, and inter-beat interval data of the one or more subjects.

15. The vital sign monitoring system of claim 14, wherein the signal processor is further configured to acquire micro-Doppler data of the region of interest.

16. The vital sign monitoring system of claim 15, wherein the signal processor is further configured to estimate the heart rate of the one or more subjects using a time-series regression of features of the micro-Doppler data.

17. The vital sign monitoring system of claim 15, wherein micro-Doppler data comprises a set of micro-Doppler images of the region of interest.

18. The vital sign monitoring system of claim 13, wherein the signal processor is further configured to use the joint motion-vital sign model to estimate a macro body motion of the one or more subjects.

19. The vital sign monitoring system of claim 18, wherein the signal processor is configured to extract the vital sign information by suppressing the macro body motion from the radar return signal.

20. The vital sign monitoring system of claim 13, wherein the signal processor is further configured to identify a first human subject in the region of interest and a second human subject in another region of interest.
